(19) United States
(12) Patent Application Publication
(10) Pub. No.: US 2001/0002264 A1
(43) Pub. Date: May 31, 2001

(54) Citrus peel extract as inhibitor of fatty streak formation on the arterial wall

(75) Inventors: Song-Hae Bok, Daejeon (KR); Tae-Sook Jeong, Daejeon (KR); Ki-Hwan Bae, Daejeon (KR); Yong-Bok Park, Daegu (KR); Myung-Sook Choi, Daegu (KR); Surk-Sik Moon, Gongju-shi (KR); Yong-Kook Kwon, Daejeon (KR); Eun-Sook Lee, Daejeon (KR); Byung-Hwa Hyun, Daejeon (KR); Yang-Kyu Choi, Daejeon (KR); Chul-Ho Lee, Daejeon (KR); Jun-Sung Lee, Daejeon (KR); Kwang-Hee Son, Daejeon (KR); Byoung-Mog Kwon, Daejeon (KR); Young-Kook Kim, Daejeon (KR); Doil Choi, Daejeon (KR); Sung-Uk Kim, Daejeon (KR); Ingyu Hwang, Daejeon (KR); Jung-Ah Ahn, Daejeon (KR); Young-Bae Park, Seoul (KR); Hyo-Soo Kim, Seoul (KR); Seong-Choon Choe, Seoul (KR)

Correspondence Address: David A. Einhorn, Esq., Anderson Kill & Olick, P.C., 1251 Avenue of the Americas, New York, NY, 10020, US

(73) Assignee: Korea Research Institute of Bioscience and Biotechnology, Daejeon (KR)

(21) Appl. No.: 09/751,101

(22) Filed: Dec. 28, 2000

Related U.S. Application Data
(63) Continuation-in-part of application No. 09/181,396, filed on Oct. 28, 1998.

(30) Foreign Application Priority Data
Oct. 28, 1997 (KR) 97-55580
Mar. 28, 1998 (KR) 98-10888
Apr. 1, 1998 (KR) 98-11450
Apr. 8, 1998 (KR) 98-12411
Apr. 14, 1998 (KR) 98-13283

(51) Int. Cl. A61K 35/78

(52) U.S. Cl. 424/736

(57) ABSTRACT

Method for inhibiting the formation of fatty streak on the arterial endothelium in a mammal comprise administering a citrus peel extract or citrus peel powder thereto.

### CROSS-REFERENCE TO RELATED APPLICATION

This application is a continuation-in-part application of co-pending U.S. Ser. No. 09/181,396 filed on Oct. 28, 1998.

### FIELD OF THE INVENTION

The present invention relates to a method for inhibiting the formation of fatty streak on the arterial endothelium in a mammal, said method comprising administering a citrus peel extract to the mammal.

### BACKGROUND OF THE INVENTION

In recent years, coronary cardio-circulary diseases, e.g., atherosclerosis and hypercholesterolemia, have increasingly become a major cause of deaths. It has been reported that an elevated plasma cholesterol level causes the deposition of fat, macrophages and foam cells on the wall of blood vessels, such deposit leading to plaque formation and then to atherosclerosis (Ross, R., Nature, 362, 801-809(1993)).

Atherosclerotic lesions are histologically clssified into six types, i.e., types I to VI by H. C. Stary et al. (Circulation, 92: 1355-1374(1995)). The initial (type I) lesion contains enough atherogenic lipoprotein to elicit an increase in macrophages and formation of scattered macrophage foam cells. As in subsequent lesion types, the changes are more marked in locations of arteries with adaptive intimal thickening. Type II lesions consist primarily of layers of macrophage foam cells and lipid-laden smooth muscle cells and include lesions grossly designated as fatty streaks. Type III is the intermediate stage between type II and type IV (atheroma, a lesion that is potentially symptom-producing). In addition to the lipid-laden cells of type II, type III lesions contain scattered collections of extracellular lipid droplets and particles that disrupt the coherence of some intimal smooth muscle cells. This extracellular lipid is the immediate precursor of the larger, confluent, and more disruptive core of extracellular lipid that characterizes type IV lesions. Beginning around the fourth decade of life, lesions that usually have a lipid core may also contain thick layers of fibrous connective tissue (type V lesion) and/or fissure, hematoma, and thrombus (type VI lesion). Some type V lesions are largely calcified (type Vb), and some consist mainly of fibrous connective tissue and little or no accumulated lipid or calcium (type Vc).

It is desirable to prevent the progression of atheroscleosis at an early stage. Accordingly, there has continued to exist a need to develop a non-toxic inhibitor of fatty streak formation on the arterial epithelium.

The present inventors have endeavored to develop a novel and potent inhibitor of fatty streak formation from natural materials, and, as a result, have discovered that citrus peel extract has a potent inhibitory activity on the fatty streak formation.

Hitherto, citrus peel has been discarded or used only for the preparation of an animal fodder or organic fertilizer. Dried citrus peel comprises 50 to 60 wt % of alcohol-insoluble polymers such as pectin, hemicellulose and cellulose; 30 to 50 wt % of alcohol-soluble solid materials (80 wt % thereof consisting of glucose, fructose and sucrose); and a small or trace amount of bioflavonoids, vitamins, limonoids, phenolic compounds and oils.

It has been reported that the bioflavonoids isolated from a citrus peel have anti-oxidative, anti-cancer, anti-viral and blood-pressure lowering activities (Saija, A., et al., Free Radical Biol. Med., 19, 481-486(1995); Matsubara, Y., et al., Japan Organic Synthesis Chem. Association Journal, 52, 318-327(1994, Mar.); Galati, E. M., et al., Farmaco, 51(3), 219-221(1996, Mar.); Felicia, V., et al., Nutr. Cancer, 26, 167-181(1996); EP 0352147 A2(1990. 1. 24); and Kaul, T. N., et al., J. Med. Viol., 15, 71-75(1985)).

However, fatty streak formation inhibitory activity of the citrus peel extract has not been reported.

### SUMMARY OF THF INVENTION

Accordingly, it is an object of the present invention to provide a method for inhibiting the formation of fatty streak on the endothelial wall of an artery in a mammal.

In accordance with one aspect of the present invention, there is provided a method for inhibiting the formation of fatty streak on the endothelial wall of an artery in a mammal which comprises administering a citrus peel extract thereto.

### BRIEF DESCRIPTION OF THE DRAWINGS

The above and other objects and features of the present invention will become apparent from the following description of the invention, when taken in conjunction with the accompanying drawings, in which:

FIGS. 1A, 1B, 1C and 1D show the arteries of the rabbits administered with 1% cholesterol; 1% cholesterol plus 1 mg/kg Lovastatin®; 1% cholesterol plus 0.1% hesperidin; and 1% cholesterol plus 0.1% naringin, respectively.

### DETAILED DESCRIPTION OF THF INVENTION

The present invention provides a method for inhibiting the formation of fatty streak on the endothelial wall of an artery in a mammal which comprises administering a citrus peel extract or citrus peel powder thereto.

The citrus may be tangerines, oranges, lemons, grapefruits, citrons, and the like. It is preferable to use peel of citrus fruits produced by organic agricultural techniques without using chemical pesticides.

The citrus peel extract of the present invention may be prepared by any of the conventional methods using suitable solvents, e.g., water, lower alcohols such as ethanol, and an aqueous alkali- or alkaline earth-metal hydroxide solution such as aqueous Ca(OH)₂ and NaOH solutions. For instance, 1 to 30 l of a solvent is added to 1 kg of dried citrus peel and the mixture is allowed to stand at a temperature ranging from 25 to 80° C. for a period ranging from 1 to 12 hours. The resulting extract is filtered and the filtrate is concentrated to obtain a concentrated peel extract. In case that an aqueous alkali- or alkalian earth-metal hydroxide solution is used, the filtrate is further adjusted to a pH ranging from 4.0 to 7.0 by adding an acid thereto, the resulting solution is allowed to stand at a temperature ranging from 1 to 10° C. for a period ranging from 10 to 48 hours, and the resulting precipitate is recovered and then dried to obtain a citrus peel extract. More preferably, 3 to 30 l of 20 to 95 % ethanol is added to 1 kg of dried citrus peel and the mixture is allowed to stand at a temperature ranging from 25 to 80° C. for a period ranging from 1 to 12 hours. The resulting extract is filtered and the filtrate is concentrated, e.g., by vacuum, to obtain a concentrated peel extract. On the other hand, 5 to 30 l of 0.1 to 2% Ca (OH)₂ or NaOH is added to 1 kg of dried citrus peel and the mixture is allowed to stand at a temperature ranging from 25 to 60° C. for a period ranging from 1 to 5 hours. The resulting extract is filtered and the filtrate is adjusted to a pH ranging from 4.0 to 7.0 by adding 1N HCl thereto. The resulting filtrate is allowed to stand at a temperature ranging from 1 to 10° C. for a period ranging from 10 to 48 hours. The resulting precipitate is recovered and then dried to obtain a citrus peel extract.

Further, a citrus peel powder may be used in the present invention in place of the citrus peel extract. The citrus peel powder may be prepared by lyophilizing or drying the solid materials including citrus peel, which remains after squeezing juice from a citrus fruit, according to a conventional method and, then, powdering it to a particle size ranging from 50 to 250 μm.

The citrus peel extract exerts an inhibitory effect on the formation of fatty streak on the endothelial wall of an artery at a dose of 1.0 mg/kg/day or more, the inhibitory effect increasing with the dose thereof.

Moreover, in spite of its potent efficacies, the citrus peel extract shows little toxicity or mitogenicity in tests using mice. More specifically, the citrus peel extract exhibits no toxicity when it is orally administered to a mouse at a dose of 1,000 mg/kg, which corresponds to an oral administration dose of 50 to 100 g of citrus peel extract for a person weighing 50 kg. Further, the citrus peel extract exerts no adverse effects on the liver function.

The present invention also provides a pharmaceutical composition for inhibiting the formation of fatty streak on the endothelial wall of an artery, which comprise a citrus peel extract as an active ingredient and pharmaceutically acceptable excipients, carriers or diluents.

A pharmaceutical formulation may be prepared in accordance with any of the conventional procedures. In preparing the formulation, the active ingredient 2% Ca (OH)₂ or NaOH is added to 1 kg of dried citrus peel and the mixture is allowed to stand at a temperature ranging from 25 to 60° C. for a period ranging from 1 to 5 hours. The resulting extract is filtered and the filtrate is adjusted to a pH ranging from 4.0 to 7.0 by adding 1N HCl thereto. The resulting filtrate is allowed to stand at a temperature ranging from 1 to 10° C. for a period ranging from 10 to 48 hours. The resulting precipitate is recovered and then dried to obtain a citrus peel extract.

Further, a citrus peel powder may be used in the present invention in place of the citrus peel extract. The citrus peel powder may be prepared by lyophilizing or drying the solid materials including citrus peel, which remains after squeezing juice from a citrus fruit, according to a conventional method and, then, powdering it to a particle size ranging from 50 to 250 μm.

The citrus peel extract exerts an inhibitory effect on the formation of fatty streak on the endothelial wall of an artery at a dose of 1.0 mg/kg/day or more, is the inhibitory effect increasing with the dose thereof

Moreover, in spite of its potent efficacies, the citrus peel extract shows little toxicity or mitogenicity in tests using mice. More specifically, the citrus peel extract exhibits no toxicity when it is orally administered to a mouse at a dose of 1,000 mg/kg, which corresponds to an oral administration dose of 50 to 100 g of citrus peel extract for a person weighing 50 kg. Further, the citrus peel extract exerts no adverse effects on the liver function.

The present invention also provides a pharmaceutical composition for inhibiting the formation of fatty streak on the endothelial wall of an artery, which comprise a citrus peel extract as an active ingredient and pharmaceutically acceptable excipients, carriers or diluents.

A pharmaceutical formulation may be prepared in accordance with any of the conventional procedures. In preparing the formulation, the active ingredient is preferably admixed or diluted with a carrier, or enclosed within a carrier which may be in the form of a capsule, sachet or other container. When the carrier serves as a diluent, it may be a solid, semi-solid or liquid material acting as a vehicle, excipient or medium for the active ingredient. Thus, the formulations may be in the form of a tablet, pill, powder, sachet, elixir, suspension, emulsion, solution, syrup, aerosol, soft and hard gelatin capsule, sterile injectable solution, sterile packaged powder and the like.

Examples of suitable carriers, excipients, and diluents are lactose, dextrose, sucrose, sorbitol, mannitol, starches, gum acacia, alginates, gelatin, calcium phosphate, calcium silicate, cellulose, methyl cellulose, microcrystalline cellulose, polyvinylpyrrolidone, water, methylhydroxybenzoates, propylhydroxybenzoates, talc, magnesium stearate and mineral oil. The formulations may additionally include fillers, anti-agglutinating agents, lubricating agents, wetting agents, flavoring agents, emulsifiers, preservatives and the like. The compositions of the invention may be formulated so as to provide quick, sustained or delayed release of the active ingredient after their administration to a mammal by employing any of the procedures well known in the art.

The pharmaceutical composition of the present invention can be administered via various routes including oral, transdermal, subcutaneous, intravenous and intramuscular introduction. In case of human, a typical daily dose of the citrus peel extract may range from about 1 to 1,000 mg/kg body weight, preferably 10 to 500 mg/kg body weight, and can be administered in a single dose or in divided doses.

However, it should be understood that the amount of the active ingredient actually administered ought to be determined in light of various relevant factors including the condition to be treated, the chosen route of administration, the age, sex and body weight of the individual patient, and the severity of the patient's symptom; and, therefore, the above dose should not be intended to limit the scope of the invention in any way.

Moreover, the citrus peel extract can be incorporated in foods or beverages, as an additive or a dietary supplement, for the purpose of inhibiting the formation of fatty streak on the arterial endothelium. The foods or beverages may include meats; juices such as a vegetable juice (e.g., carrot juice and tomato juice) and a fruit juice (e.g., orange juice, grape juice, pineapple juice, apple juice and banana juice); chocolates; snacks; confectionery; pizza; foods made from cereal flour such as breads, cakes, crackers, cookies, biscuits, noodles and the likes; gums; dairy products such as milk, cheese, yogurt and ice creams; soups; broths; pastes, ketchups and sauces; teas; alcoholic beverages; carbonated beverages such as Coca-Cola® and Pepsi-Cola®; vitamin complexes; and various health foods.

In this case, the content of the citrus peel extract in a food or beverage may range from 0.5 to 10% by weight. In particular, the beverage according to the present invention may comprise 10 to 100 g of the citrus peel extract per 1,000 ml of the beverage. In case of citrus peel powder, the content thereof in a food or beverage may range from 0.5 to 30% by weight.

As described above, the citrus peel extract or citrus peel powder can be used as an effective, non-toxic pharmaceutical agent for inhibiting the formation of fatty streak on the arterial endothelium.

The following Examples are intended to further illustrate the present invention without limiting its scope.

Further, percentages given below for solid in solid mixture, liquid in liquid, and solid in liquid are on a wt/wt, vol/vol and wt/vol basis, respectively, and all the reactions were carried out at room temperature, unless specifically indicated otherwise.

### EXAMPLE 1

### Preparation and Analysis of Citrus Peel Extract

The peels of tangerines (Cheju Island, Korea), citrons (Jeollanamdo, Korea), and oranges, grapefruits and lemons (California, Calif., U.S.A.) were dried at a room temperature and powdered to a particle size ranging from 100 to 200 μm. 50 ml of methanol was added to 500 mg each of the citrus peel powder and extracted in a water bath at 50° C. for 6 hours. The extract thus obtained was cooled and filtered, and then methanol was added to the filtrate to a volume of50 ml.

To confirm the composition of the citrus peel extract obtained above, 5.0 μl of the resulting extract was subjected to high performance liquid chromatography (HPLC) using Lichrosorb® RP-8 column (5 μm, 4×250 mm) which was pre-equilibrated with 37% methanol and maintained at a temperature of 30° C. The extract was eluted with 37% methanol at a flow rate of 1.0 ml/min. Standard solutions were prepared by dissolving hesperidin and naringin (Sigma Chemical Co. U.S.A.) in methanol to final concentrations of 0.1, 0.2, 0.3, 0.4 and 0.5 mg/ml, respectively, and subjected to HPLC under the same condition as above. The eluates were detected at 280 nm with UV-VIS spectrophotometer and the contents of hesperidin and naringin were calculated by comparing the areas of HPLC profiles of the citrus peel extract and the standard solution. The contents (%) of hesperidin and naringin in various citrus peel extracts are shown in Table I.

TABLE I

| | Hesperidin (%) | Naringin (%) |
|---|---|---|
| Orange | 2.10 | trace amount |
| Lemon | 1.40 | trace amount |
| Tangerine | 2.10 | trace amount |
| grapefruit | — | 4.70 |
| citron | 0.80 | 0.80 |

### EXAMPLE 2

### Preparation of Citrus Peel Extract

(1) Method using Ethanol

The peel of tangerine (Cheju island, Korea) was dried at a room temperature and 5 l of 30% ethanol was added to 500 g of the dried peel. The peel was extracted at 60° C. for 5 hours. The extract thus obtained was filtered through cotton cloths and the filtrate was concentrated under vacuum to obtain 190 g of syrupy extract. The content of hesperidin in the citrus peel extract were examined in accordance with the method of Example 1 and it was discovered that the citrus peel extract contains 5.1 g of hesperidin.

Further, the composition of the citrus peel extract was confirmed by HPLC and the result is shown in Table II.

TABLE II

| Ingredient | | Content (%) |
|---|---|---|
| Moisture | | 65 |
| Free | Fructose | 11 |
| saccharides | Glucose | 11 |
| | Sucrose | 6 |

| | |
|---|---|
| Hesperidin | 2.7 |
| Others | 4.3 |

(2) Method using Ca(OH)₂

The peel of tangerine (Cheju island, Korea) was dried at a room temperature and 5 l of 0.5% Ca (OH)₂ solution was added to 500 g of the dried peel. The peel was extracted at a room temperature for 1 hour while stirring and the extract thus obtained was filtered through cotton cloths. 1N HCl solution was added to the filtrate to adjust its pH to 4.5. The same procedure as above was repeated to obtain a filtrate except that pH of the filtrate was adjusted to pH 6.8. The filtrates thus obtained were allowed to stand at 5° C. for 24 hours. The precipitates thus obtained were recovered and dried to obtain 5 g and 10 g of powders, respectively. HPLC analysis of the powers demonstrated is that the citrus peel extracts contained 3.2 g and 6.55 g of hesperidin (purity: 64% and 65%), respectively.

(3) Method using NaOH

The peel of tangerine (Cheju island, Korea) was dried at a room temperature and 5 l of 0.5% NaOH was added to 500 g of the dried peel.

The peel was extracted at a room temperature for 1 hour while stirring and the extract thus obtained was filtered through cotton cloths. 1N HCl solution was added to the filtrate to adjust its pH to 4.5. The same procedure as above was repeated to obtain a filtrate except that pH of the filtrate was adjusted to pH 6.8. The filtrates thus obtained were allowed to stand at 5° C. for 24 hours. The precipitates thus obtained were recovered and dried to obtain 44 g and 49 g of powders, respectively. HPLC analysis of the powers demonstrated that the citrus peel extracts contained 13.9 g and 9.8 g of hesperidin (purity: 31% and 20%), respectively.

### EXAMPLE 3

### Toxicity of Orally Administered Citrus Peel Extract

7 to 8 week-old, specific pathogen-free ICR female mice (6 heads) each weighing about 25 to 29 g and male mice (6 heads) each weighing about 34 to 38 g were bred under a condition of temperature 22±1° C., moisture 55±5% and photoperiod 12L/12D. Fodder (Cheiljedang Co., mouse and rat fodder) and water were sterilized and fed to the mice.

The citrus peel extract obtained in Example 2(1) was dissolved in 0.5% Tween® 80 to a concentration of 100 mg/ml, and the solution was orally administered to the mice in an amount of 0.2 ml per 20 g of mouse body weight. The solution was administered once and the mice were observed for 10 days for signs of adverse effects or death according to the following schedule: 1, 4, 8, and 12 hours after the administration and, every 12 hours thereafter. The weight changes of the mice were recorded every day to examine the effect of citrus peel extract. Further, on the 10th day, the mice were sacrificed and the internal organs were visually examined.

All the mice were alive at day 10 and the citrus peel extract showed no toxicity at a dose of 1,000 mg/kg. The autopsy revealed that the mice did not develop any pathological abnormality, and no weight loss was observed during the 10 day test period. Accordingly, it was concluded that the citrus peel extract is not toxic when orally administered to an animal.

### EXMAPLE 4

### Inhibition of Formation of Fatty Streak on the Endothelial Wall of an Artery in Citrus Peel Extract-Fed Animals

(Step 1) Administration of citrus bioflavonoids to animals

36 three-month-old New Zealand White rabbits (Yeonam Horticulture and Animal Husbandry College, Korea) each weighing about 2.5 to 2.6 kg were bred under a condition of temperature 20±2° C., relative humidity 55±5%, and photoperiod 12L/12D. The rabbits were divided by a group of 6 rabbits, and is the rats of six groups were fed with six different diets, i.e., RC4 diet (Oriental Yeast Co., Japan) containing 1% cholesterol (Control group); 1% cholesterol plus 1 mg/kg Lovastatin® (Merck, U.S.A.) (Comparative group); 1% cholesterol plus 0.1% hesperidin; 1% cholesterol plus 0.1% hesperetin; 1% cholesterol plus 0.1% naringin; and 1% cholesterol plus 0.1% naringenin, respectively. RC4 diet comprises 7.6% moisture, 22.8% crude protein, 2.8% crude fat, 8.8% crude ash, 14.4% crude cellulose and 43.6% soluble nitrogen-free substances. The rabbits were bred for 6 weeks while being allowed free access to the diets and water.

(Step 2) Analysis for fatty streak in the main artery

The rabbits bred in (Step 1) were sacrificed and their chest were incised. The main artery was cut out therefrom in a length of about 5 cm downward from the site 1 cm above the aortic valve and the fat surrounding the main artery was removed. The main artery was incised in the middle along the longitudinal axis and pinned to a dish. The moist artery was photographed and, then, staining of fatty streak was carried out in accordance with the method of Esper, E., et al. (J. Lab. Clin. Med., 121, 103-110(1993)) as follows.

A part of the incised main artery was washed three times by 2 min. with anhydrous propylene glycol and stained for 30 min. with a saturated solution of Oil Red O (ORO, Sigma Co.) dissolved in propylene glycol. Thereafter, the artery was washed twice by 3 min. with 85% propylene glycol to remove remaining staining solution and, then washed with physical saline. The artery was photographed and the photograph was traced. The area of stained region (fatty streak region) was determined with an image analyzer (LEICA, Q-600, Germany) and its proportion (%) to the total arterial area was calculated.

On the other hand, the other part of the main artery was stained in accordance with hematoxylin-eosin (H&E) and Masson's trichrome staining methods and observed under a microscope to confirm whether the fatty streaks were accumulated in the intima, intemus, elastic lamina and media.

Further, blood samples were taken from the rats of the six dietary groups and plasma HDL fractions were separated therefrom by using HDL-cholesterol reagent (Sigma Chemical Co., Cat. No. 352-3) containing dextran-sulfate. Total cholesterol level was determined by using Sigma Diagnostic Kit Cat. No. 352-100(Sigma Chemical Co., U.S.A.). Triglyceride level was determined by using Sigma Diagnostic Kit Cat. No. 339-50.

The result is shown in Table III.

TABLE III

| | Total Cholesterol | Triglyceride | Fatty Streak |
|---|---|---|---|
| Dietary Group | (mg/dl) | (mg/dl) | Area (%) |
| Control group | 1143 | 56 | 35 |
| Lovastatin group | 1210 | 66 | 5 |
| Hesperidin group | 1130 | 40 | 13.5 |
| Hesperetin group | 1150 | 41 | 13 |
| Naringin group | 1367 | 72 | 12 |
| Naringenin group | 1350 | 70 | 13 |

As can be seen from Table III, the area of fatty streak accumulated on the arterial endothelium decreased significantly in the Lovastatin®, hesperidin, hesperetin, naringin and naringenin groups, as compared to the control group. Accordingly, it has been confirmed that hesperidin, hesperetin, naringin and naringenin isolated from citrus peel extract, as well as citrus peel extract containing the flavonoids, inhibit the formation of fatty streak on the arterial endothelium. In particular, it is remarkable that the inhibitory activity of the bioflavonoids isolated from citrus peel extract on the formation of fatty streak was exhibited under the blood cholesterol levels above 1,100 mg/dl, which are much higher than that of normal rabbit, i.e., about 50 mg/dl. This result suggests that there may be a novel mechanism for preventing the onset of atherosclerosis, which is different from the blocking of cholesterol synthesis by a HMG-CoA reductase inhibitor, blocking of cholesterol absorption by an ACAT inhibitor, or blocking of cholesterol transfer by a CETP inhibitor.

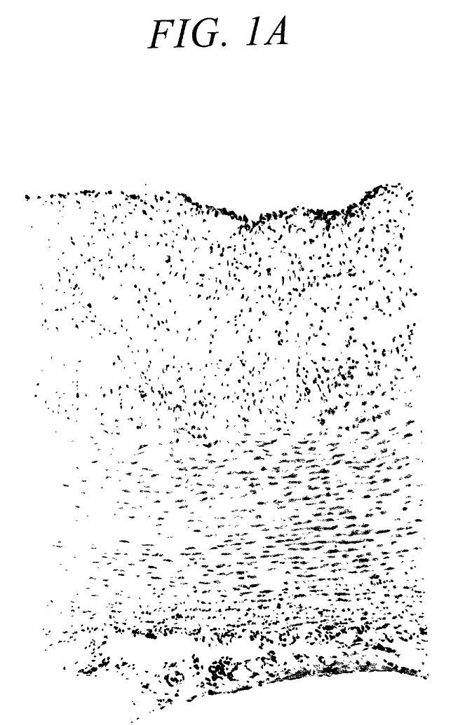

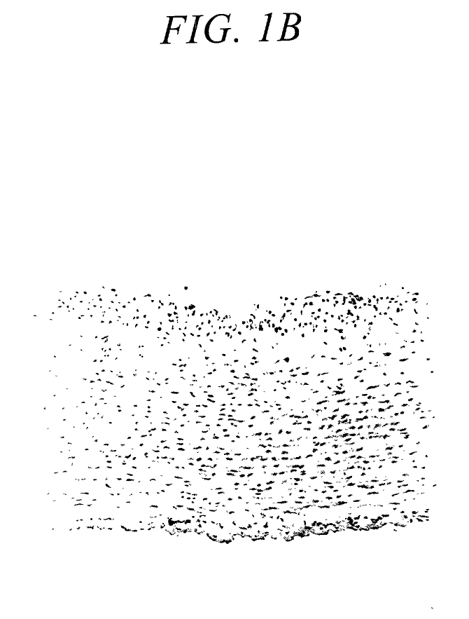

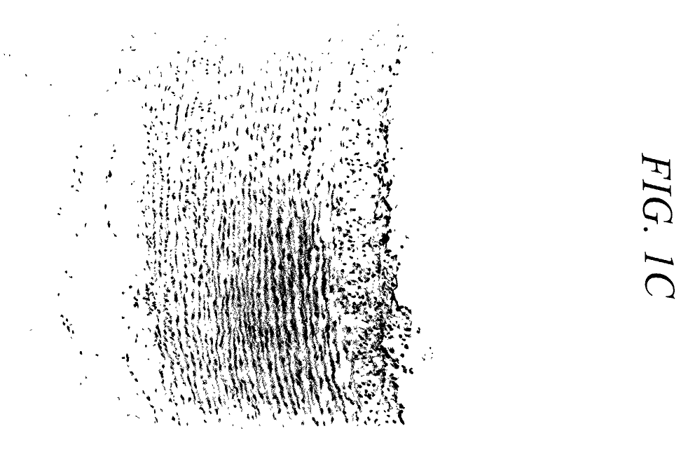

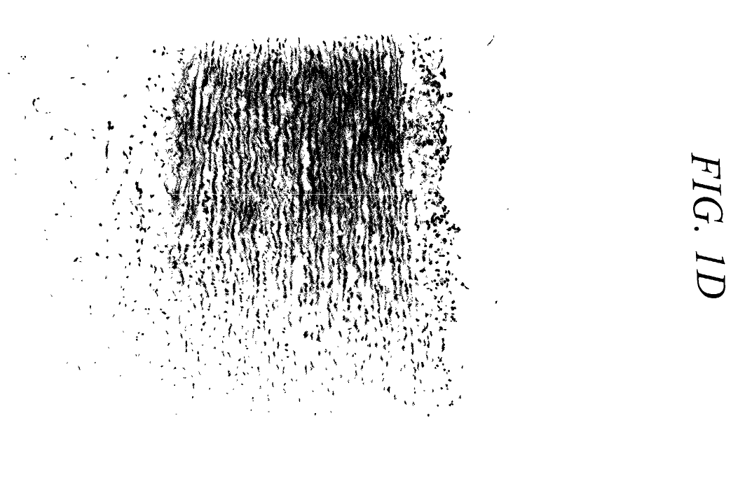

FIGS. 1A, 1B, 1C and 1D show the arteries of the rabbits administered with 1% cholesterol (control group); 1% cholesterol plus 1 mg/kg Lovastatin® (comparative group); 1% cholesterol plus 0.1% hesperidin; and 1% cholesterol plus 0.1% naringin, respectively. As shown in FIGS. 1A, 1B, 1C and 1D, a thick layer of fatty streak was observed on the arterial endothelium of the rabbit administered with 1% cholesterol, while no or very thin layers of fatty streak were observed on the arterial endotheliums of the rabbits administered with 1% cholesterol plus 1 mg/kg Lovastatin®, 1% cholesterol plus 0.1% hesperidin, and 1% cholesterol plus 0.1% naringin, respectively.

Accordingly, it has been concluded that citrus bioflavonoids such as hesperidin, hesperetin, naringin and naringenin, as well as citrus peel extract containing them strongly inhibit the formation of fatty streak on the arterial endothelium.

### EXAMPLE 5

### Pharmaceutical Preparation Containing Citrus Peel Extract

A soft capsule was prepared using the following ingredients:

| | Quantity (mg/capsule) |
|---|---|
| Citrus peel extract of Example 2(1) | 20 |
| Starch, dried | 160 |
| Magnesium stearate | 20 |
| Total | 200 |

The above ingredients were mixed thoroughly, and the resulting mixture was filled into a soft gelatin capsule in accordance with a conventional capsule preparation method to obtain a soft gelatin capsule preparation.

### EXAMPLE 6

### Foods Containing Citrus Peel Powder or Extract

Foods containing citrus peel powder or extract obtained in Examples 1 and 2 were prepared as follows.

(1) Preparation of tomato ketchup and sauce

The citrus peel powder obtained in Example 1 was added to a tomato ketchup or sauce in an amount ranging from 1 to 20 wt % to obtain a health-improving tomato ketchup or sauce. Alternatively, the citrus peel extract obtained in Example 2 (1) was added to a tomato ketchup or sauce in an amount ranging from 0.5 to 10 wt % to obtain a health-improving tomato ketchup or sauce.

(2) Preparation of wheat flour foods

The citrus peel powder obtained in Example 1 was added to a wheat flour in an amount ranging from 1 to 30 wt % and breads, cakes, cookies, crackers and noodles were prepared by using the mixture to obtain health-improving foods.

Alternatively, these foods were prepared by using a wheat flour containing 0.5 to 10 wt % of the citrus peel extract obtained in Example 2 (1).

(3) Preparation of soups and gravies

The citrus peel powder obtained in Example 1 was added to soups and gravies in an amount ranging from 1 to 30 wt % to obtain health-improving soups and gravies.

Alternatively, these foods were prepared by using soups and gravies containing 0.5 to 10 wt % of the citrus peel extract obtained in Example 2 (1).

(4) Preparation of ground beef

The citrus peel powder obtained in Example 1 was added to ground beef in an amount ranging from 1 to 30 wt % to obtain a health-improving ground beef.

Alternatively, these foods were prepared by using ground beef containing 0.5 to 10 wt % of the citrus peel extract obtained in Example 2 (1).

(5) Preparation of dairy product

The citrus peel powder obtained in Example 1 or citrus peel extract obtained in Example 2 (1) was added to milk in an amount ranging from 0.5 to 10 wt % and various dairy products such as butter and ice cream were prepared by using the milk.

However, in case of cheese preparation, the citrus peel powder or extract was added to the coagulated milk protein; and, in case of yogurt preparation, the citrus peel powder or extract was added to the coagulated milk protein obtained after the fermentation.

### EXMAPLE 7

### Beverages containing Citrus Peel Powder or Extract

(1) Preparation of vegetable juice

10 to 100 g of the citrus peel powder obtained in Example 1 or citrus peel extract obtained in Example 2 (1) was added to 1000 ml of a tomato or carrot Juice to obtain a health-improving vegetable juice.

(2) Preparation of fruit juice

10 to 100 g of the citrus peel powder obtained in Example 1 or citrus peel extract obtained in Example 2 (1) was added to 1000 ml of an apple or grape Juice to obtain a health-improving fruit juice.

(3) Preparation of carbonated drink

1 to 100 g of the citrus peel powder obtained in Example 1 or citrus peel extract obtained in Example 2 (1) was added to 1000 ml of Coca-Cola® or Pepsi-Cola® to obtain a health-improving carbonated drink.

While the invention has been described with respect to the above specific embodiments, it should be recognized that various modifications and changes may be made to the invention by those skilled in the art which also fall within the scope of the invention as defined by the appended claims.

What is claimed is:

1. A method for inhibiting the formation of fatty streak on the arterial endothelium in a mammal which comprises administering an effective amount of a citrus peel extract or powder thereto.

2. The method of claim 1, wherein the mammal is human.

3. The method of claim 1, wherein the citrus is tangerines, oranges, lemons or grapefruits.

4. The method of claim 1, wherein the effective amount of the citrus peel extract ranges from 1 to 1,000 mg/kg body weight/day.

5. The method of claim 1, wherein the effective amount of the citrus peel powder ranges from 1 to 1,000 mg/kg body weight/day.

6. The method of claim 1, wherein the citrus peel extract is prepared by extracting dried citrus peel with a solvent selected from the group consisting of water, a lower alcohol and an aqueous alkali- or alkaline earth-metal hydroxide solution.

7. The method of claim 6, wherein the citrus peel extract is prepared by a process including the steps of: adding 3 to 30 l of 20 to 95% ethanol to 1 kg of dried citrus peel; allowing the mixture to stand at a temperature ranging from 25 to 80° C. for a period ranging from 1 to 12 hours; filtering the resulting extract; and concentrating the filtrate to obtain the citrus peel extract.

8. The method of claim 6, wherein the citrus peel extract is prepared by a process including the steps of: adding 5 to 30 l of 0.1 to 2% Ca (OH)₂ or NaOH to 1 kg of dried citrus peel; allowing the mixture to stand at a temperature ranging from 25 to 60° C. for a period ranging from 1 to 5 hours; filtering the resulting extract; adjusting the filtrate to a pH ranging from 4.0 to 7.0; allowing the resulting filtrate to stand at a temperature ranging from 1 to 10° C. for a period ranging from 10 to 48 hours; and, recovering and drying the resulting precipitate to obtain the citrus peel extract.

9. The method of claim 1, wherein the citrus peel powder is prepared by a process including the steps of: lyophilizing or drying the solid materials remaining after squeezing juice from citrus fruits; and powdering the dried materials to a particle size ranging from 50 to 250 μm.

10. The method of claim 1, wherein the citrus peel extract or citrus peel powder is administered in the form of a pharmaceutical composition containing an effective amount of the citrus peel extract and a pharmaceutically acceptable carrier.

11. The method of claim 1, wherein the citrus peel extract or citrus peel powder is administered in the form of an additive or a dietary supplement in food or beverage.

12. The method of claim 11, wherein the content of the citrus peel extract in the food ranges from 0.5 to 10% by weight.

13. The method of claim 11, wherein the content of the citrus peel powder in the food ranges from 1 to 30% by weight.

14. The method of claim 11, wherein the food is meats, chocolates, snacks, confectionery, pizza, foods made from cereal flour, gums, dairy products, soups, broths, pastes, ketchups, sauces, vitamin complexes or health foods.

15. The method of claim 14, wherein the foods made from cereal flour is breads, cakes, crackers, cookies, biscuits or noodles.

16. The method of claim 11, wherein the beverage is dairy products, vegetable juices, fruit juices, teas, alcoholic beverages or carbonated beverages.

17. The method of claim 11, wherein the content of the citrus peel extract in the beverage ranges from 10 to 100 g per 1,000 ml of the beverage.
